(19) United States
(12) Patent Application Publication
(10) Pub. No.: US 2024/0059756 A1
(43) Pub. Date: Feb. 22, 2024

(54) METHOD FOR PRODUCING HUMAN COLLAGEN STRUCTURES WITH CONTROLLED CHARACTERISTICS

(71) Applicant: José Adán ORTEGA BLANCO, Zapopan, Jalisco (MX)

(72) Inventors: Juan Pablo AGUILAR ALEMÁN, Zapopan, Jalisco (MX); Beni CAMACHO PÉREZ, Zapopan, Jalisco (MX); Brenda Karen AGUILLÓN ESTRADA, Zapopan, Jalisco (MX); Grecia Andrea CARDOSO HERNÁNDEZ, Zapopan, Jalisco (MX); Octavio Israel ROJAS GARCÍA, Zapopan, Jalisco (MX); Eduardo MIRAMONTES BEAS, Zapopan, Jalisco (MX)

(73) Assignee: José Adán ORTEGA BLANCO, Zapopan, Jalisco (MX)

(21) Appl. No.: 18/267,990

(22) PCT Filed: Mar. 11, 2021
(86) PCT No.: PCT/MX2021/000007

§ 371 Date: Jun. 16, 2023

(30) Foreign Application Priority Data
Dec. 18, 2020 (MX) MX/A/2020/014328

(51) Int. Cl. C07K 14/78; C07K 1/36; B01D 61/24

(52) U.S. Cl. CPC C07K 14/78; C07K 1/36; B01D 61/243; B01D 2325/0283; B01D 2311/12

(57) ABSTRACT

A method for producing human collagen structures with controlled characteristics, having the following stages: a) Tissue conditioning b) Pre-treatment; b1) final washings with distilled water; c) extraction by enzymatic hydrolysis by subjecting the tissue to a solution of acetic acid with pepsin; d) precipitation, where the resulting collagen solution is brought to a high concentration by adding sodium chloride and where the fibers recovered from the sieve are solubilized again in an acetic acid solution; e) dynamic dialysis to purify the solution from the excess of salt present in the collagen solution; f) lyophilization at −40° C. and a vacuum pressure of 0.04 mbar (4 Pa); g) molding or second lyophilization, where a concentration is chosen and collagen is solubilized in an acetic acid solution; once solubilized, collagen is placed in molds to generate the desired structure and once again, the solution is lyophilized; h) crosslinking, where the collagen pieces are subjected to a formaldehyde vapor atmosphere in a crosslinking apparatus and i) pressing, where the collagen structure is subjected to a mechanical force of 400-5000 N compacting to a value of 0.01-10 mm and increasing its fibrillar density.

### FIELD OF THE INVENTION

The present invention is related to the field of biotechnology in general; in particular, it is related to a novel process for the production of human collagen structures with controlled characteristics, which allows good performance while preserving structural integrity and high purity.

### BACKGROUND OF THE INVENTION

Collagen is one of the most abundant essential proteins in the human body, it represents 25% of the total body protein, which is part of different tissues: it provides flexibility, elasticity and resistance to pressure.

This protein is very abundant in skin, bones, connective tissues and joints (ligaments, tendons and cartilage). It is also found in the cornea and in the walls of blood vessels. Collagen is an essential protein for the proper functioning of the body.

Type I collagen is by far the most abundant collagen in the body. It has an unusual amino acid composition, with 33% glycine and 10% proline. It also has 0.5% 3-hydroxyproline, 10% 4-hydroxyproline, and 1% 5-hydrolysine. These hydroxylated amino acids are post-translationally synthesized from prolyl and lysyl residues in the polypeptide. Collagen has a small amount of carbohydrates, most of them attached to the hydroxyl group of hydroxylisine in the form of Glu-Gal disaccharide. Carbohydrate content in fibrillar collagens is low (0.5-1% in types I and II), but is higher in some non-fibrillar types (14% in type IV). Meisenberg, G. and Simmmon, W. Principles of Medical Biochemistry, 4th Edition.

Type I collagen has the special feature of providing flexibility and elasticity, it is the most abundant in the body, mainly in the bones, tendons, skin and cornea; while type II collagen provides resistance to pressure and is mainly located in cartilage.

Collagen is used extensively in cosmetic surgery and in the construction of artificial skin grafts for the treatment of patients with severe burns. Collagen products are also used in orthopedic and surgical applications in a wide range of dental procedures. Human collagen is derived from cadaveric tissues or debris from surgical procedures, such as the placenta at the time of childbirth; and it has the advantage of significantly reducing the probability of an immune reaction, compared to collagen from other species.

Due to the importance of collagen in several medical and industrial applications, processes have been developed to produce human collagen structures.

There are industrial processes that can produce similar results in terms of obtaining collagen. However, no process has been found that can specifically obtain type I human collagen, with a yield of 35%. Nor has a process been found that can transform the human collagen obtained in varied two-dimensional or three-dimensional structures with controllable characteristics. Nor has a process been found that can precisely control the characteristics of collagen.

Other processes for obtaining collagen are also known that comprise defined stages already widely known by those who are engaged in this area. The stages, in general, include a pretreatment of raw material, hydrolysis and purification of collagen. There are processes in which certain operating parameters or stages have been slightly modified to extract collagen from several animal sources, such as cattle, pigs, horses, birds and marine animals; using the skin, scales, bones, tendons, pericardium, cartilage, etc., of said animals.

Methods of collagen production in vitro, through the use of cell lines, are also known.

Other known methodologies describe an acidic or basic pretreatment of raw material, for example, with NaOH at different concentrations, temperatures and exposure times.

There are also hydrolysis techniques for obtaining soluble collagen in several acids, or through enzymatic treatments. Organic acids such as acetic acid, lactic acid, citric acid and the like have been used for chemical hydrolysis; or inorganic acids such as hydrochloric acid; while proteolytic enzymes such as ficin, pepsin, among others, are reported for enzymatic hydrolysis. Similarly, these techniques are operated in various ranges of operating parameters of temperature, time, pH and concentration of the solution and enzyme.

Other processes are those designed to obtain and purify several types of collagen from the same source, by means of fractional precipitation techniques, at different concentrations of salts such as NaCl, ammonium sulfate and the like, or with ethanol.

Methods for collagen purification and concentration, such as dialysis and lyophilization, are also known.

There are methods for producing defined structures of collagen, such as scaffolds, sponges, membranes, coatings, hydrogels, etc., through lyophilization, gelation, electrospinning, bioprinting, among others.

Physical and chemical methods have been described for reinforcing the biopolymer chains and improving the stability and rigidity of the structure, as well as for controlling the degradation rate in vivo and in vitro. As regards these methods, we can mention dehydrothermal treatment, ultraviolet radiation, chemical crosslinking with aldehydes such as glutaraldehyde and formaldehyde in solution, the use of carbodiimides and microbial transglutaminase.

Similarly, there are compression processes for collagen structures, particularly hydrogels, to modify the mechanical properties and fibrillar density of the collagen structure.

The present invention is focused on a novel process for the production of human collagen structures with controlled characteristics, which allows good performance while preserving structural integrity and high purity.

A search was carried out to determine the closest state of the art, and the following documents were found.

Patent document U.S. Pat. No. 4,389,487 was found; this document is by Ries; Peter E. of Jun. 8, 1981, and it discloses a process for the preparation of collagen products for medical and cosmetic use that comprises the stages of finely grinding animal tissues containing collagen at temperatures not exceeding 40° C., degreasing the resulting tissue pulp and simultaneously removing undesirable components thereof by repeatedly treating said tissue pulp with approximately a five-fold volume, based on pulp volume, of a 5 to 15% aqueous solution of sodium chloride containing about 0.2 to 1 part by weight of sodium azide as a preservative per 1000 parts by weight of said solution and 0.5 to 2% by weight of a non-ionic fat-dispersing wetting agent, washing the resulting fiber pulp with water or with 0.1 to 0.5% aqueous formic, acetic or citric acid, digesting the fiber pulp for 8 to 48 hours at a pH of about 2.5 to 3.5 in a five-fold volume, based on the pulp volume, of 0.1 to 5% of aqueous acetic acid containing about 1 part by weight of pepsin per 1000 parts by weight of tissue used as a starting material for removing non-collagenous proteins and telopeptides, precipitating collagen from the resulting collagen suspension by the addition of aqueous sodium chloride in an amount such that the sodium chloride concentration of the suspension is approximately 3 to 5%, separating the precipitated collagen and substantially desalting it by ultrafiltration, dialysis or washing with 60 to 75% aqueous ethyl alcohol so that the collagen concentration in the desalted solution is approximately 1%, dissolve the desalted collagen in demineralized or distilled water containing up to 3% by weight of a volatile strong organic acid in such a proportion that the collagen concentration in the resulting solution corresponds to a dry residue of approximately 0.5 to 2% by weight, freeze-dry the collagen solution and sterilize the lyophilized collagen product, the improvement comprising: subjecting the collagen product to a heat treatment or gaseous hydrogen halide treatment after lyophilization, before or after sterilization, for a time sufficient to impart increased absorption and wet strength of the collagen product.

The objective of U.S. Pat. No. 4,389,487 is the improvement of the physicochemical and mechanical properties of collagen products for their handling during use. In addition, increasing the absorption capacity. The use of a hydrogen chloride atmosphere in the treatment of a collagen product managed to improve the physicochemical and mechanical properties, adding that an absorption of 50 to 100 times its weight was obtained. Compared with the methodology proposed in our invention is the use of a controlled atmosphere of paraformaldehyde to improve physicochemical properties; thus, an absorption of 60 times its weight was achieved. In addition, it should be noted that the proposal of this application is the use of scraps of tendons, fascia lata, amniotic membrane, fat or skin of human origin compared to U.S. Pat. No. 4,389,487 where one of its claims is the use of pig skin and bovine tendons.

U.S. Pat. No. 4,389,487 as described above, discloses a process for the preparation of collagen products for medical and cosmetic use that is highly complex, it requires many reagents and variable controls in different percentages; furthermore, the resulting collagen product is subjected to a heat treatment or gaseous hydrogen halide treatment after lyophilization, before or after sterilization, for a time sufficient to impart increased absorption and wet strength of the collagen product.

Document WO2016071876 by Fontanilla Duque, Martha Raquel, et al. of Nov. 6, 2015, was also found; this document discloses a method to isolate type I collagen from the rat tail and produce multidirectional scaffolds (Perez et al., 2001). In recent years, the isolation method has been modified and standardized. Likewise, changes have been introduced in the method of obtaining scaffolds from this collagen and a novel method has been developed to produce unidirectional lamellar scaffolds. In the optimized method, the animal collagen source is cleaned, cut and suspended in an acetic acid solution until a liquid phase pH between 2.5 and 3.2 is reached, stirring constantly at 4° C. Said suspension is centrifuged and the pH of the supernatant obtained is neutralized with NaOH to separate the solid and liquid phases again. Collagen precipitated in this way is reprecipitated with salts and solubilized in acetic acid, and the solution is poured into molds, frozen at −20° C. and lyophilized. Finally, the scaffolds obtained are crosslinked and then sterilized with ethylene oxide.

The process for obtaining rat collagen is a chemical process compared to the method proposed in our application, which integrates chemical and biological processes (use of pepsin) to obtain the collagen scaffold of human origin. Another difference, in patent WO2016071876, the crosslinking process uses different crosslinking agents (genipin, glutaraldehyde, dendrimers, carbodiimides or a mixture of them) compared to paraformaldehyde, which is the crosslinking agent in our invention. Furthermore, they use ethylene oxide to carry out the final sterilization compared to the process of our invention which uses gamma irradiation.

On the one hand, with the collagen extraction processes described in the literature, although they explore the use of different collagen sources, in the vast majority it is not possible to obtain profitable extraction yields under large-scale conditions, or they are not available in great demand; the most used raw materials for the industrial production of collagen are usually bovine or porcine tissues. However, collagen structures derived from these sources present the risk of eliciting an immune response during their application, since, being a xenograft, biocompatibility decreases.

On the other hand, the possibility of elaborating collagen structures with defined and very specific structural and dimensional characteristics, which contribute to a better integration of the structures in an in vivo environment, influencing cell behavior, has been addressed through techniques that can be very expensive, not very accessible or adaptable on a large scale, or that require specialized equipment, such as electrospinning.

Similarly, despite the fact that collagen is an excellent structural protein and has the advantages of being biocompatible and resorbable, its mechanical properties regarding synthetic polymers are poor. Various methods and techniques have been developed to improve these features and diversify the use of collagen structures; however, said techniques have important disadvantages, such as not being efficient, being processes that require a lot of time, but if they are extended too much, they can denature collagen; some crosslinking techniques or agents can cause a crosslinking gradient along the entire collagen structure, generating a structure with heterogeneous mechanical properties; the addition of some chemical components can result in a cytotoxic effect, and said compounds can remain in the collagen structure even after extensive washing; to mention a few.

### OBJECTIVES OF THE INVENTION

The main objective of the present invention is to make available a process to produce human collagen structures with controlled characteristics that allows to obtain type I human collagen, with a yield of 35%, preserving structural integrity and with purity ≥95%.

Another objective of the invention is to provide said process for producing human collagen structures with controlled characteristics, which also offers the possibility of producing collagen structures with defined and very specific structural and dimensional characteristics, which contribute to a better integration of the structures in a in vivo environment, influencing cell behavior.

Another objective of the invention is to provide said process to produce human collagen structures with controlled characteristics, which also allows the use of an available, accessible raw material with the highest degree of biocompatibility; obtaining functional and structurally intact collagen, with profitable extraction yields through a compatible process for industrial production conditions.

Another objective of the invention is to provide said process to produce human collagen structures with controlled characteristics, which also allows the elaboration of collagen structures with adjustable physical characteristics for a wide range of biological applications, by means of simple and low-cost techniques that allow the optimization of collagen concentration in the structures; such characteristics include the dimensions of the structure, fibrillar density, porosity and pore size, which strongly influence cell behavior.

Another objective of the invention is to provide said process for producing human collagen structures with controlled characteristics, which also allow the mechanical performance of the structures to be increased, with a precise and controlled technique, without affecting the integrity, functionality, or biological safety of collagen.

Another objective of the invention is to provide said process to produce human collagen structures with controlled characteristics, which allows it to be a support or vehicle for the culture of primary and line eukaryotic cells, as well as a deposit for growth factors, proteins and exosomes, for possible use in regenerative medicine.

And all those qualities and objectives that will become apparent when making a general and detailed description of the present invention supported by the illustrated modalities.

### BRIEF DESCRIPTION OF THE INVENTION

The process to produce human collagen structures with controlled characteristics ranges from isolating tissue to obtaining collagen, as well as its subsequent transformation into bi- or three-dimensional structures that can be applied in multiple medical procedures. It consists of a total of 8 main stages: tissue conditioning, pre-treatment, extraction, precipitation, dialysis, lyophilization, molding and crosslinking, as well as an optional compression stage.

Likewise, it is intended to protect the entire process itself. From the extraction to the adaptation of the substrate (collagen) to a regenerative device. Our process allows the extraction of allogeneic type 1 collagen, and thereby it can be chemically conditioned (cross-linked) to achieve in vivo functional characteristics. Finally, this last process allows it to be adapted to be able to get ready and thereby achieve the desirable in vitro and in vivo degradation conditions.

In general terms, the process to produce human collagen structures with controlled characteristics, in accordance with the present invention, consists of the following stages:
a) Tissue conditioning, where tissues or fluids that are not of interest for the process are removed and it is reduced to a particle size of 0.5 to 1 mm in a uniform way, in order to achieve a better interaction between solutions and tissue in the subsequent steps.
b) Pre-treatment

Exposure of the previously conditioned tissue to a solution of sodium hydroxide at a mold concentration of 0.05-2 M, using 100 mL per gram of dry tissue, with efficient magnetic agitation and for a time of 2-6 hours, in order to remove surface proteins and leave collagen fibers exposed;
b1) carry out a series of final washes with distilled water, which allow neutralizing the pH of the tissue to a pH value between 7-8, before proceeding with the process.
c) Extraction where enzymatic hydrolysis is performed by subjecting the tissue to an acid solution of acetic acid at a molar concentration of 0.02-0.5 M and using 450 L per kg of dry tissue, together with 200 g of pepsin per kg of dry tissue (2.27% pepsin in 0.5 M acetic acid) to promote a faster solubilization of collagen and the specific and controlled removal of the terminal carboxyl and amino groups of the molecule; where the extraction is carried out over a period of three days by adding intermediate acid solution, starting with 200 mL of acetic acid per g of dry tissue and after 48 hours 250 mL of acetic acid per g of dry tissue are added, to promote the development of the reaction and where the residues of non-collagenous tissue structures are removed by means of filtration and the process continues.
d) Precipitation, where the resulting collagen solution (450 mL of solution per g of dry tissue) is brought to a high salt concentration by adding sodium chloride at a rate of 58.44 g of sodium chloride per L of collagen solution, the solution is homogenized with magnetic stirring and the ionic interaction of the salt with the collagen molecules is allowed to generate their precipitation in a time of about 2-6 hours. The resulting solution is sieved to a particle size between 125-850 μm; wherein the fibers recovered from the sieve are solubilized again in an acetic acid solution using 350 mL of acetic acid per g of dry tissue;
e) Dialysis, where in order to purify the solution from the excess of salt present in the collagen solution, a dynamic dialysis system is employed by means of which collagen is placed inside a porous membrane with a pore size of between 12-14 kD that allows the expulsion of impurities when introduced into a dialysis buffer consisting of a solution with a low concentration of acid acetic from 0.02-0.5 M; where the exchange of salt molecules between both solutions originates from their concentration differential, from the collagen solution with a salt concentration of 1-2 M towards the acetic acid solution without the presence of salts and is accelerated by the contact surface and the buffer flow; this stage lasts three to four days, during which the buffer is kept in recirculation and is changed after 48 hours; the conductivity of both solutions is also monitored and the process is stopped when collagen reaches the same conductivity as the buffer at the beginning of the stage of a value between 0.20-0.28 mS/cm.
f) Lyophilization; where the purified collagen solution is subjected to a lyophilization cycle to concentrate the collagen fibers, favoring their preservation; in order to achieve this, the solution is frozen to −40° C. and subsequently subjected to a vacuum pressure of 0.04-0.2 mbar (4-20 Pa) for a period of two days, during which water and solvents are removed in steam form drying the collagen without damaging the fibers; where the resulting collagen is weighed and distributed according to what is required in the next step.
g) Molding or second lyophilization, where depending on the application and expected function, a concentration of between 2.5-10 mg/mL is chosen and collagen is solubilized again in an acetic acid solution; where between 2.5-10 mg of collagen per mL of acetic acid with a molarity of between 0.02-0.5 M are used; once solubilized, collagen is placed in molds that allow to generate the desired structure; once again, the solution is lyophilized at a temperature of −40° C. and a vacuum pressure of between 0.04-0.2 mbar (4-20 Pa), with the exception that a controlled freezing is carried out (which consists of removing heat by gradually lowering the temperature of the plate with which the mold and collagen solution are in contact, allowing the water and acetic acid crystals to be uniform and varied, accommodating the fibers) to generate an estimated average pore size;
h) crosslinking, where the collagen pieces are subjected to a formaldehyde vapor atmosphere in a crosslinking apparatus, which allows the exposure time to be set between 1-60 minutes and the concentration of the reagent vapor cloud between 0.2-1.6 ppm resulting in a controlled crosslinking that allows reinforcing the bond between fibers that provides better physical properties to the structure.

In one of the modalities of the invention, the process also includes the stage of:
i) Pressing (or additional stage according to the application), where, in relation to the application and function to be performed by the product, where the collagen structure is subjected to a determined mechanical force of 400-5000 N to compact its dimensions to a desired value from 0.01-10 mm and increase its fibrillar density.

The human collagen structures obtained with controlled characteristics are type I human collagen, with a yield of 35%, preserving structural integrity and with purity ≥95%.

The human collagen structures obtained by the described process are also characterized in that they allow the cultivation and co-cultivation of primary and/or line human cells, and they function as a deposit of growth factors, proteins and exosomes, for their possible use in regenerative therapy.

In order to better understand the characteristics of the present invention, the present description is accompanied, as an integral part thereof, by the drawings with an illustrative but non-limiting nature, which are described below.

### BRIEF DESCRIPTION OF THE FIGURES

FIG. 1 shows a flow chart of the novel process for the production of human collagen structures with controlled characteristics, in accordance with the present invention.

For a better understanding of the invention, there will be a detailed description of some of its modalities, shown in the drawings that are attached to this description for illustrative but not limiting purposes.

### DETAILED DESCRIPTION OF THE INVENTION

The characteristic details of the novel process for the production of human collagen structures with controlled characteristics are clearly shown in the following description and in the attached illustrative drawings, which provide reference signs to indicate the same steps.

During the development of the novel process for the production of human collagen structures with controlled characteristics, a series of problems arose that were gradually resolved until the process was standardized to obtain type I human collagen structures with controlled characteristics, with a yield of 35%, preserving structural integrity and with purity ≥95%.

The process to reduce the particle size of tissues was time consuming and involved a lot of manipulation. Besides that irregular pieces were obtained. It was solved by choosing to process the tissue in grinding equipment that would allow obtaining a paste from the tissue, without applying too much mechanical stress that would degrade its structure. In order to maintain a low temperature during the process and to avoid the degradation of the extracted collagen fibers, a refrigerator with internal connection was adapted for the equipment used throughout the process.

Due to the amount of solution and the size of the fibers obtained after fractional precipitation, a sieving separation was implemented, which reduced the operation time and eliminated the need to acquire a larger and more specialized equipment to carry out separation by centrifugation of larger volumes of solution, resulting in only a 4% loss of fibers.

To enhance the mechanical performance and resistance to enzymatic degradation of the collagen structures and enable the final structure to adequately perform its intended function, chemical crosslinking of the structure was proposed; this operation falls within the field of bioconjugation.

In this context, chemical crosslinking should be understood as the creation of covalent bonds between two or more molecules. When carrying out this process, the result should be a structure with greater mechanical resistance, greater resistance to deformation, longer enzymatic degradation time and greater absorption. Different chemical substances that could achieve this effect were investigated and it was decided to try glutaraldehyde and formaldehyde.

Firstly, a solution of water and glutaraldehyde was used to immerse the structure in it. This method was unfeasible due to the delicate nature of the structure, which prevented the overall shape from being maintained, although it mainly affected the thickness and ductility of the material, making it highly brittle. Glutaraldehyde was simply too aggressive on the collagen structure, collapsing it while crosslinking it. The same technique was tried with a solution of water and formaldehyde, but the results were still unacceptable.

Afterwards, the decision was made to generate a gaseous medium in which the collagen structures were placed to interact at the molecular level with the formaldehyde gas. This medium or atmosphere was created simply by allowing the mixture of water and formaldehyde to remain for some time in a closed container at room temperature. The aqueous solution, due to the evaporation that naturally occurs at room temperature, eventually saturated the volume of the container. After a specified time, the container was opened and the collagen structures were introduced. Since the structures were inside the container, a specific time began to be counted. This method was equally unfeasible due to several factors: on the one hand, the water and formaldehyde solution did not evaporate in a determinable or controllable manner. In addition, when opening the container to introduce the collagen structures, the equilibrium reached by the atmosphere inside was disturbed.

This alteration could not be easily determined or measured. Ultimately, this method could not reliably generate repeatable results.

It was assumed that using mixtures of formaldehyde with some other medium (such as water), would make it more difficult to control the amount of gas generated. Therefore, the decision was made to produce the purest gas possible. It was demonstrated that it was feasible to heat paraformaldehyde until reaching the sublimation temperature and generate formaldehyde gas. Once the gas has been produced, it should be passed to another closed chamber where the collagen structures had previously been placed on a support that would allow homogeneous interaction with the gas. It was also decided that a certain amount of vacuum be produced in the chamber to minimize the effect that atmospheric air might have on the crosslinking process. Another issue that had to be resolved was how to stop the process abruptly in order to control both the start and the end of the crosslinking. To achieve this, a method was conceived to include a medical-grade air intake that would serve to purge the system and remove the formaldehyde.

By removing the formaldehyde, the interaction between collagen molecules and formaldehyde molecules is stopped, effectively stopping the crosslinking process. All of these design requirements were generated based on observation and experiments as described in the previous paragraphs. So the final design of the apparatus is the result of an experimental process and it is also the solution that allows collagen structures to be achieved with the desired physical, mechanical, chemical and biological compatibility characteristics.

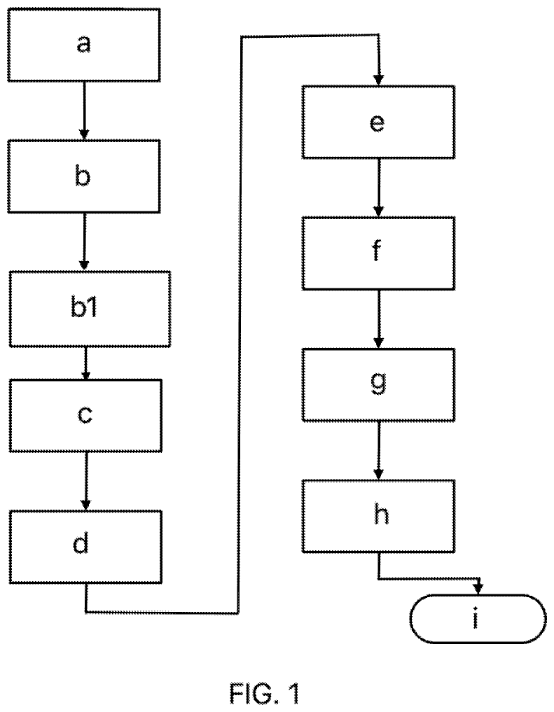

According to FIG. 1, the novel process for the production of human collagen structures with controlled characteristics, in accordance with the present invention, consists of the following stages:
a) Tissue conditioning, where tissues or fluids that are not of interest for the process are removed and it is reduced to a particle size of 0.5 to 1 mm in a uniform way, in order to achieve a better interaction between solutions and tissue in the subsequent steps.
b) Pre-treatment

Exposure of the previously conditioned tissue to a solution of sodium hydroxide at a mold concentration of 0.05-2 M, using 100 mL per gram of dry tissue, with efficient magnetic agitation and for a time of 2-6 hours, in order to remove surface proteins and leave collagen fibers exposed;
b1) carry out a series of final washes with distilled water, which allow neutralizing the pH of the tissue to a pH value between 7-8, before proceeding with the process.
c) Extraction where enzymatic hydrolysis is performed by subjecting the tissue to an acid solution of acetic acid at a molar concentration of 0.02-0.5 M and using 450 L per kg of dry tissue, together with 200 g of pepsin per kg of dry tissue (2.27% pepsin in 0.5 M acetic acid) to promote a faster solubilization of collagen and the specific and controlled removal of the terminal carboxyl and amino groups of the molecule; where the extraction is carried out over a period of three days by adding intermediate acid solution, starting with 200 mL of acetic acid per g of dry tissue and after 48 hours 250 mL of acetic acid per g of dry tissue are added, to promote the development of the reaction and where the residues of non-collagenous tissue structures are removed by means of filtration and the process continues.
d) Precipitation, where the resulting collagen solution (450 mL of solution per g of dry tissue) is brought to a high salt concentration by adding sodium chloride at a rate of 58.44 g of sodium chloride per L of collagen solution, the solution is homogenized with magnetic stirring and the ionic interaction of the salt with the collagen molecules is allowed to generate their precipitation in a time of about 2-6 hours. The resulting solution is sieved to a particle size between 125-850 μm; wherein the fibers recovered from the sieve are solubilized again in an acetic acid solution using 350 mL of acetic acid per g of dry tissue;
e) Dialysis, where in order to purify the solution from the excess of salt present in the collagen solution, a dynamic dialysis system is employed by means of which collagen is placed inside a porous membrane with a pore size of between 12-14 kD that allows the expulsion of impurities when introduced into a dialysis buffer consisting of a solution with a low concentration of acid acetic from 0.02-0.5 M; where the exchange of salt molecules between both solutions originates from their concentration differential, from the collagen solution with a salt concentration of 1-2 M towards the acetic acid solution without the presence of salts and is accelerated by the contact surface and the buffer flow; this stage lasts three to four days, during which the buffer is kept in recirculation and is changed after 48 hours; the conductivity of both solutions is also monitored and the process is stopped when collagen reaches the same conductivity as the buffer at the beginning of the stage of a value between 0.20-0.28 mS/cm.
f) Lyophilization; where the purified collagen solution is subjected to a lyophilization cycle to concentrate the collagen fibers, favoring their preservation; in order to achieve this, the solution is frozen to −40° C. and subsequently subjected to a vacuum pressure of 0.04-0.2 mbar (4-20 Pa) for a period of two days, during which water and solvents are removed in vapor form drying the collagen without damaging the fibers; where the resulting collagen is weighed and distributed according to what is required in the next step.
g) Molding or second lyophilization, where depending on the application and expected function, a concentration of between 2.5-10 mg/mL is chosen and collagen is solubilized again in an acetic acid solution; where between 2.5-10 mg of collagen per mL of acetic acid with a molarity of between 0.02-0.5 M are used; once solubilized, collagen is placed in molds that allow to generate the desired structure; once again, the solution is lyophilized at a temperature of −40° C. and a vacuum pressure of between 0.04-0.2 mbar (4-20 Pa), with the exception that a controlled freezing is carried out (which consists of removing heat by gradually lowering the temperature of the plate with which the mold and collagen solution are in contact, allowing the water and acetic acid crystals to be uniform and varied, accommodating the fibers) to generate an estimated average pore size;
h) crosslinking, where the collagen pieces are subjected to a formaldehyde vapor atmosphere in a crosslinking apparatus, which allows the exposure time to be set between 1-60 minutes and the concentration of the reagent vapor cloud between 0.2-1.6 ppm resulting in a controlled crosslinking that allows reinforcing the bond between fibers that provides better physical properties to the structure.

In one of the modalities of the invention, the process also includes the stage of:
i) Pressing (or additional stage according to the application), where, in relation to the application and function to be performed by the product, where the collagen structure is subjected to a determined mechanical force of 400-5000 N to compact its dimensions to a desired value from 0.01-10 mm and increase its fibrillar density.

With the production process, it has been possible to use of an available, accessible raw material with the highest degree of biocompatibility; obtaining functional and structurally intact collagen, with profitable extraction yields through a compatible process for industrial production conditions. In addition, said process allows the elaboration of collagen structures with adjustable physical characteristics for a wide range of biological applications, by means of simple and low-cost techniques that allow the optimization of collagen concentration in the structures; such characteristics include the dimensions of the structure, fibrillar density, porosity and pore size, which strongly influence cell behavior.

Likewise, it was possible to increase the mechanical performance of the structures, with a precise and controlled technique, without affecting the integrity, functionality, or biological safety of collagen.

The human collagen structures obtained by the described process are also characterized in that they allow the cultivation and co-cultivation of primary and/or line human cells, and they function as a deposit of growth factors, proteins and exosomes, for their possible use in regenerative therapy.

The invention has been sufficiently described so that a person with average knowledge in the field can reproduce and obtain the results mentioned in the present invention. However, any person skilled in the relevant technical field of this invention may be capable of making modifications not described in the present application; nevertheless, if the matter claimed in the following claims is required for the implementation of these modifications in a specific structure or in the manufacturing process thereof, such structures must be included within the scope of the invention.

1. A method to produce human collagen structures with controlled characteristics, characterized by comprising the following stages:
a) tissue conditioning, where tissues or fluids that are not of interest for the method are removed and it is reduced to a particle size of 0.5 to 1 mm in a uniform way, in order to achieve a better interaction between solutions and tissue in the subsequent steps;
b) pre-treatment
exposure of the previously conditioned tissue to a solution of sodium hydroxide at a mold concentration of 0.05-2 M, using 100 mL per gram of dry tissue, with efficient magnetic agitation and for a time of 2-6 hours, in order to remove surface proteins and leave collagen fibers exposed;
b1) carry out a series of final washes with distilled water, which allow neutralizing the pH of the tissue to a pH value between 7-8, before proceeding with the method;
c) Extraction where enzymatic hydrolysis is performed by subjecting the tissue to an acid solution of acetic acid at a molar concentration of 0.02-0.5 M and using 450 L per kg of dry tissue, together with 200 g of pepsin per kg of dry tissue (2.27% pepsin in 0.5 M acetic acid) to promote a faster solubilization of collagen and the specific and controlled removal of the terminal carboxyl and amino groups of the molecule; where the extraction is carried out over a period of three days by adding intermediate acid solution, starting with 200 mL of acetic acid per g of dry tissue and after 48 hours 250 mL of acetic acid per g of dry tissue are added, to promote the development of the reaction and where the residues of non-collagenous tissue structures are removed by means of filtration and the method continues;
d) precipitation, where the resulting collagen solution (450 mL of solution per g of dry tissue) is brought to a high salt concentration by adding sodium chloride at a rate of 58.44 g of sodium chloride per L of collagen solution, the solution is homogenized with magnetic stirring and the ionic interaction of the salt with the collagen molecules is allowed to generate their precipitation in a time of about 2-6 hours, the resulting solution is sieved to a particle size between 125-850 μm; wherein the fibers recovered from the sieve are solubilized again in an acetic acid solution using 350 mL of acetic acid per g of dry tissue;
e) dialysis, where in order to purify the solution from the excess of salt present in the collagen solution, a dynamic dialysis system is employed by means of which collagen is placed inside a porous membrane with a pore size of between 12-14 kD that allows the expulsion of impurities when introduced into a dialysis buffer consisting of a solution with a low concentration of acid acetic from 0.02-0.5 M; where the exchange of salt molecules between both solutions originates from their concentration differential, from the collagen solution with a salt concentration of 1-2 M towards the acetic acid solution without the presence of salts and is accelerated by the contact surface and the buffer flow; this stage lasts three to four days, during which the buffer is kept in recirculation and is changed after 48 hours; the conductivity of both solutions is also monitored and the method is stopped when collagen reaches the same conductivity as the buffer at the beginning of the stage of a value between 0.20-0.28 mS/cm;
f) lyophilization; where the purified collagen solution is subjected to a lyophilization cycle to concentrate the collagen fibers, favoring their preservation; in order to achieve this, the solution is frozen to −40° C. and subsequently subjected to a vacuum pressure of 0.04-0.2 mbar (4-20 Pa) for a period of two days, during which water and solvents are removed in vapor form drying the collagen without damaging the fibers; where the resulting collagen is weighed and distributed according to what is required in the next step;
g) molding or second lyophilization, where depending on the application and expected function, a concentration of between 2.5-10 mg/mL is chosen and collagen is solubilized again in an acetic acid solution; where between 2.5-10 mg of collagen per mL of acetic acid with a molarity of between 0.02-0.5 M are used; once solubilized, collagen is placed in molds that allow to generate the desired structure; once again, the solution is lyophilized at a temperature of −40° C. and a vacuum pressure of between 0.04-0.2 mbar (4-20 Pa), with the exception that a controlled freezing is carried out (which consists of removing heat by gradually lowering the temperature of the plate with which the mold and collagen solution are in contact, allowing the water and acetic acid crystals to be uniform and varied, accommodating the fibers) to generate an estimated average pore size;
h) crosslinking, where the collagen pieces are subjected to a formaldehyde vapor atmosphere in a crosslinking apparatus, which allows the exposure time to be set between 1-60 minutes and the concentration of the reagent vapor cloud between 0.2-1.6 ppm resulting in a controlled crosslinking that allows reinforcing the bond between fibers that provides better physical properties to the structure.

2. The method to produce human collagen structures with controlled characteristics, according to claim 1, characterized in that it also comprises the stage of:
i) pressing wherein relation to the application and function to be performed by the product, where the collagen structure is subjected to a determined mechanical force of 400-5000 N to compact its dimensions to a desired value from 0.01-10 mm and increase its fibrillar density.

3. The method to produce human collagen structures with controlled characteristics, according to claim 2, characterized in that the pressing generates fine and highly dense collagen structures.

4. The method to produce human collagen structures with controlled characteristics, according claim 1, characterized in that it generates a yield of 35% collagen.

5. The method to produce human collagen structures with controlled characteristics, according to claim 1, characterized in that collagen has a purity ≥95%.

6. The method to produce human collagen structures with controlled characteristics, according to claim 1, characterized in that the collagen obtained allows the cultivation and co-cultivation of primary and/or line human cells; likewise, it functions as a deposit of growth factors, proteins and exosomes, for its possible use in regenerative therapy.
